(19) United States
(12) Patent Application Publication
(10) Pub. No.: US 2012/0186005 A1
(43) Pub. Date: Jul. 26, 2012

(54) Lens to protect the eyes of a wearer

(76) Inventor: Kenneth K. Anderson, Newport Beach, CA (US)

(21) Appl. No.: 12/931,084

(22) Filed: Jan. 25, 2011

(51) Int. Cl. A61F 9/02; A61F 9/00; A41D 13/11

(52) U.S. Cl. 2/452; 2/426; 2/9; 2/15

(57) ABSTRACT

A protective lens to be attached to a face mask or used without a face mask as goggles or glasses to cover the eyes of, for example, one playing the game of paintball. The protective lens has a body to be worn over the eyes of a wearer and a thermal backing that is spaced from the lens body to lie closer to the wearer's eyes than the lens body. A volume of air or similar gas is trapped within an air space between the lens body and the thermal backing to prevent the transfer of heat from one to the other. A seal surrounds the air space so that the air trapped therein is isolated from the atmosphere. A rib projecting from the lens body surrounds and lies outside the seal to prevent the seal from becoming saturated with liquid, paint, and the like which might be encountered during play. The trapped air within the air space prevents the protective lens from fogging and obstructing the wearer's view as a consequence of the lens body and the thermal backing having different temperatures.

### BACKGROUND OF THE INVENTION

1. Field of the Invention

This invention relates to a protective lens to be carried by a face mask or used as goggles or glasses independent of a face mask and positioned across the eyes of a wearer (e.g., one playing the game of paintball). The protective lens includes a rib that surrounds an air space located between the lens body and a thermal backing within which air is trapped to prevent fogging of the viewing area through the lens.

2. Background Art

The game of paintball is spreading in popularity among players of all ages. During the game, paintballs are propelled at high speed from a paintball marker (i.e., gun) towards an opponent with the object of striking the opponent and leaving a tell-tale paint mark when the paintball ruptures on impact. So as to prevent injury to the face, it is common for players to wear protective lenses during play. However, such conventional eye-covering lenses are characterized by several shortcomings.

Because of the environment in which the protective lens is worn during the game of paintball, the viewing area through the lens can become fogged. That is to say, the outside of the protective lens has a temperature which may differ from the temperature at the inside of the lens that lies against the face of the player. Such a temperature differential is known to cause the lens to fog and obstruct the wearer's line of sight through the viewing area towards a target or other point of interest. In this case, because of the short time during which to react to the actions of an opponent, a player whose vision is obstructed is at an increased risk of being hit with a paintball. In this same regard, the player can spend time in the middle of play to wipe the protective lens or even remove the lens from his head in order to clear the obstructed viewing area or substitute a different lens. However, the expenditure of such time will also expose the player to an added risk of being hit by the opponent's paintball.

Dual pane lenses are known having outer and inner layers that are affixed one above the other by means of a seal lying therebetween. Because the seal is exposed to the environment during play, it often becomes saturated with liquid which, over time, can cause the seal to degrade. For example, paintballs which strike the lens while traveling at very high speed are capable of causing the seal to split on impact and/or leaving the seal covered by thick paint. A seal failure allows water, paint and paintball debris to leak between the layers which may ultimately result in the layers delaminating and the entire lens having to be scrapped and replaced. What is even more, a player sometimes uses his fingernail to tamper with the seal as the lens is being positioned on or removed from his face.

### SUMMARY OF THE INVENTION

In general terms, a protective lens is disclosed having particular application to cover and protect the eyes of one playing the game of paintball. The protective lens can be carried at the front of a face mask that is worn over the player's head or held against the player's face and across the eyes independently of a face mask so as to function as either goggles or glasses. The protective lens includes a generally arcuate wrap-around optically-transparent body that extends from one ear of the wearer to the other ear. Optional attachment holes are formed in the opposite sides of the lens body for receipt of connectors by which the lens can be attached to a headstrap.

According to a preferred embodiment, a rib is molded into the protective lens so as to project outwardly from the lens body towards the eyes of the wearer. The rib surrounds the viewing area through the lens which lies in the line of sight from the eyes of the wearer to a target or other point of interest. A narrow, resilient (e.g., foam) seal is located around the viewing area of the lens so as to lie inside and against the rib. One side of the seal is attached (e.g., adhesively bonded) to the lens body. An optically-transparent thermal backing is attached (e.g., adhesively bonded) to the opposite side of the seal so that the lens body and the thermal backing are separated from one another, and a space is established therebetween over the viewing area of the lens.

In the preferred embodiment, air is trapped in the space between the lens body and the thermal backing. However, different optically-transparent insulators, such as a gas other than air, can occupy the space. The seal surrounds the space between the lens body and the thermal backing to isolate the trapped air from the atmosphere. By virtue of the air trapped within the space, a temperature differential between the lens body and the thermal backing during play will not cause the lens body to fog and obscure the viewing area through the protective lens. As an important advantage of this invention, the rib against which the seal is located safeguards and prevents the seal from becoming saturated with paint as a consequence of an exploding paintball or by liquids and debris encountered within the environment during play. Hence, the life of the seal can be advantageously prolonged to prevent a delamination of the thermal backing from the lens body along the seal.

### BRIEF DESCRIPTION OF THE DRAWINGS

FIG. 1 shows a protective lens according to a first preferred embodiment carried by a face mask to be worn over the head so that the lens covers the eyes of the wearer;

FIG. 2 shows the protective lens according to another preferred embodiment to be attached to a headstrap and used as goggles over the wearer's eyes;

FIG. 3 is a perspective view of the protective lens separated from the face mask of FIG. 1 and the headstrap of FIG. 2;

FIG. 4 is an exploded view of the protective lens of FIG. 3;

FIG. 5 is a cross-section of the protective lens taken along lines 5-5 of FIG. 3; and

FIG. 6 shows a pair of glasses having a protective lens according to yet another preferred embodiment of this invention.

### DESCRIPTION OF THE PREFERRED EMBODIMENT

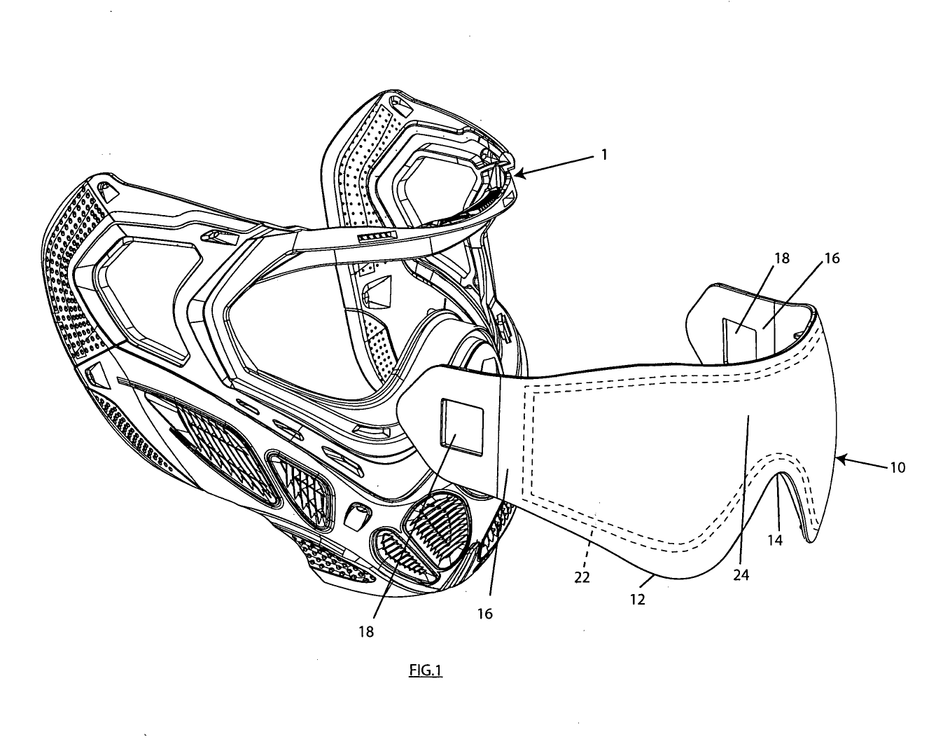

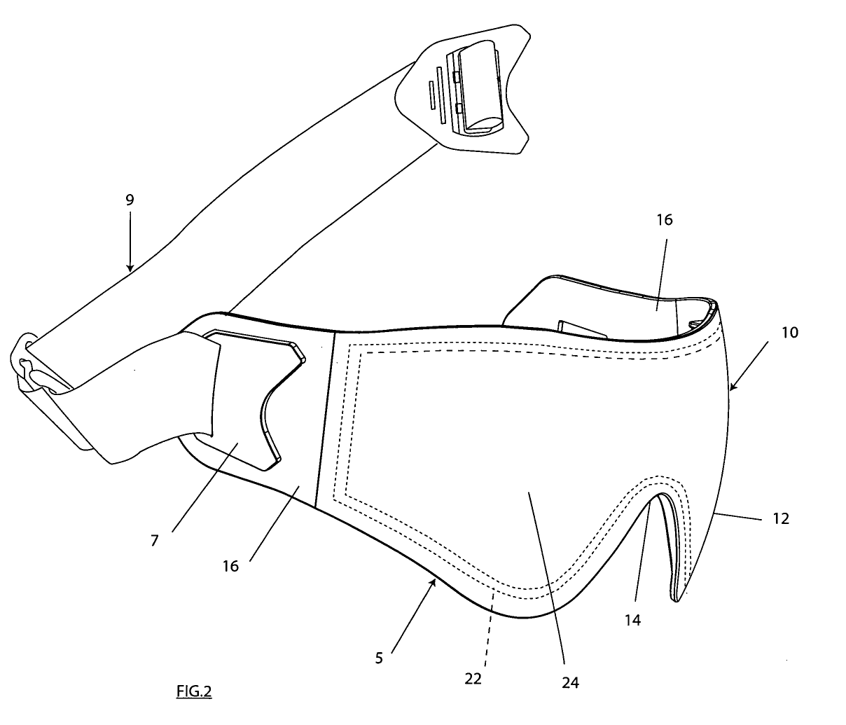

The protective lens 10 of the present invention to cover and protect the eyes of a wearer is initially described while referring concurrently to FIGS. 1-5 of the drawings. In a first preferred embodiment, the lens 10 herein disclosed has particular application to be attached to and carried by a face mask 1 (best shown in FIG. 1) of the kind to be worn over the head of one playing the game of paintball. By way of example only, reference can be made to patent application Ser. No. 11/705,121 filed Feb. 12, 2007 for details of a suitable face mask similar to that shown in FIG. 1 with which the lens 10 can be used. Nevertheless, the specific type of face mask to which the lens 10 is attached and the application of such face mask are not to be considered limitations of this invention. In fact, for some applications (e.g., skiing) the lens 10 can be worn on the player's head and over the eyes independently of a face mask. In this case, and as another preferred embodiment, the protective lens can be used as goggles 5 (best shown in FIG. 2).

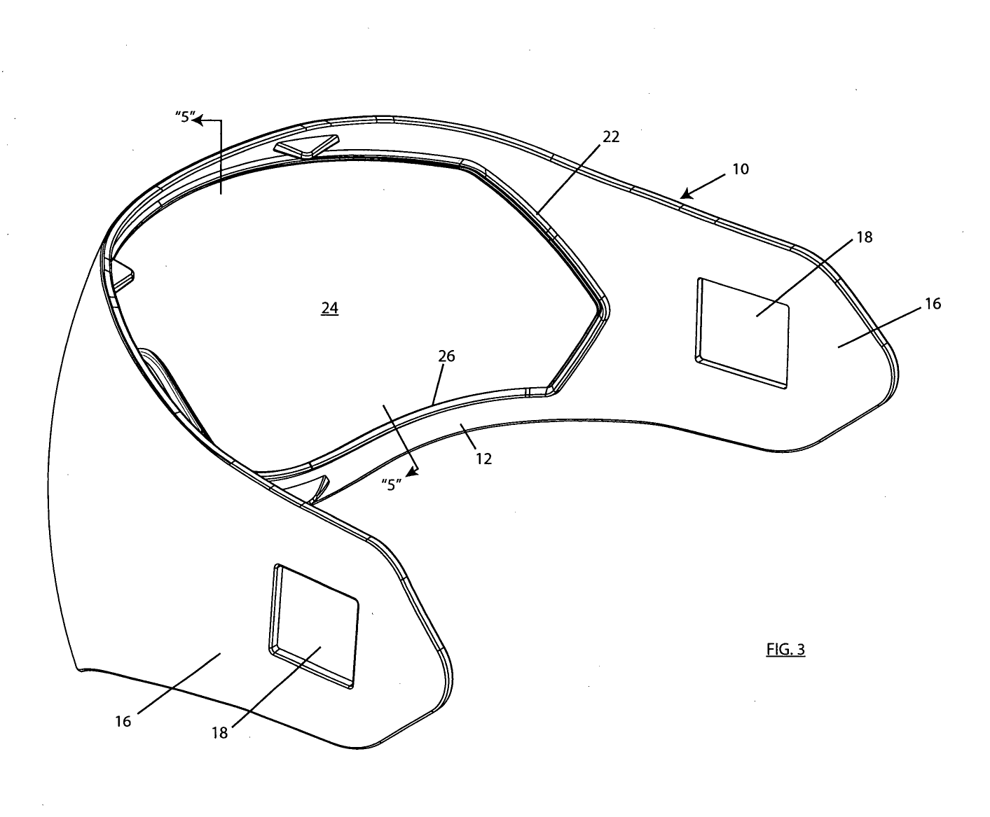

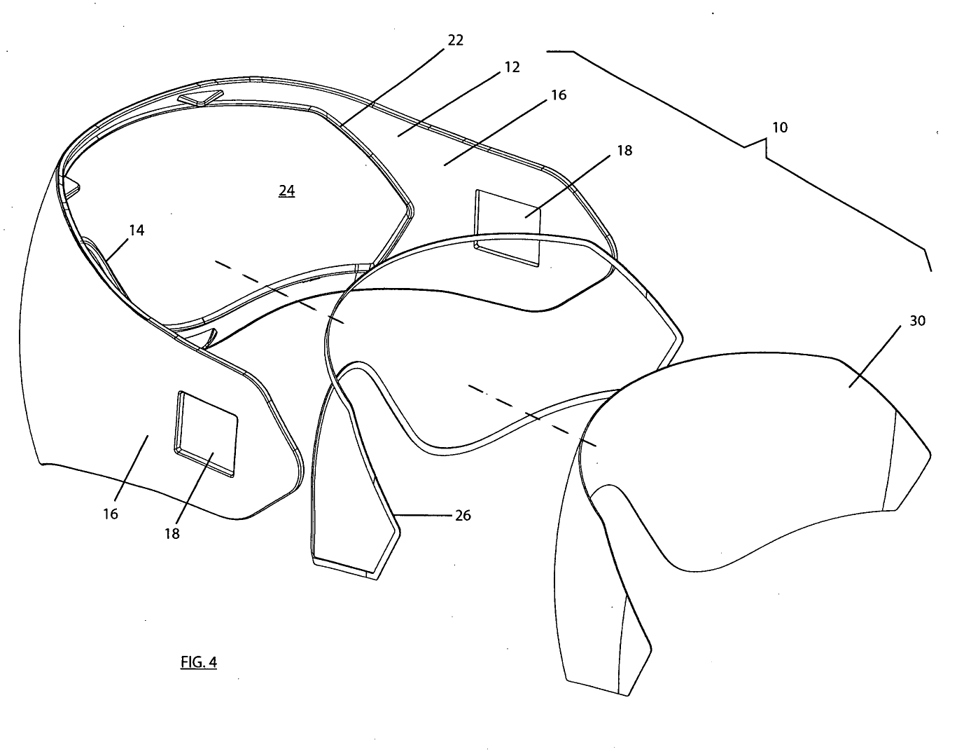

As is best shown in FIGS. 3 and 4, the protective lens 10 is of the wrap-around type having an arcuate body 12 that extends across the wearer's eyes from a location adjacent one ear, over the nose, to a location adjacent the other ear. The body 12 of lens 10 is preferably manufactured from an optically-transparent flexible material such as polycarbonate, or the like. An upturned notch or recess 14 is formed in the middle of the body 12 and shaped to surround the bridge of the wearer's nose. When the lens 10 is attached to a face mask (designated 1 in FIG. 1), opposite sides 16 of the body 12 will lie above respective ones of the wearer's ears. An optional attachment hole 18 is formed in each of the sides 16 of the lens body 12. Each attachment hole 18 is sized to be able to receive therethrough a connector (designated 7 in FIG. 2) so that the lens 10 can be detachably connected to a headstrap 9 that is sized to fit around the back of the head of the wearer to hold the lens 10 against the wearer's face. At least one such connector 7 is connected to each of the opposite ends of the headstrap 9.

In accordance with a preferred embodiment, a rib 22 is molded into the lens 10 so as to stand outwardly from the lens body 12 towards the eyes of the wearer. The rib 22 runs continuously around the lens 10 between the attachment holes 18 through the sides 16 of the body 12. The rib 22 surrounds a viewing area 24 of the lens 10 through which a line of sight is established between the wearer's eyes covered by the lens and a target or similar point of interest.

A narrow seal 26 is attached (e.g., adhesively bonded) to the lens 10 so as to lie inside the rib 22 that projects from the lens body 12. To this end, the seal 26 is surrounded by and seated against the rib 22. The seal 26 is preferably manufactured from a resilient material, such as foam, or the like.

A thermal backing 30 is attached to and spaced from the body 12 of the lens 10 by the seal 26 which lies inside the rib 22. To accomplish the aforementioned attachment of the thermal backing 30, the seal 26 is ideally covered with an adhesive on each side thereof. A first of the adhesive covered sides of the seal 26 is pressed against the lens 10 around the viewing area 24 thereof so that the seal is bonded to the lens body 12 to create a fluid-tight seal along the rib 22. The outside edge of the thermal backing 30 is pressed against the opposite adhesive covered side of the seal 26 so that the thermal backing is attached to the lens 10 along the seal. The thermal backing 30 has an arcuate shape with a curvature to match the curvature of the arcuate lens body 12. The thermal backing 30 is preferably manufactured from an optically-transparent plastic material that is substantially insensitive to changes in temperature. That is, the shape and dimensions of the thermal backing 30 will undergo little change in response to a change of temperature. Thermal backing 30 may be manufactured, for example, from the same material (i.e., polycarbonate) as the lens body 12 or a different suitable material that will not obstruct the user's line of sight through the viewing area 24 of the lens 10.

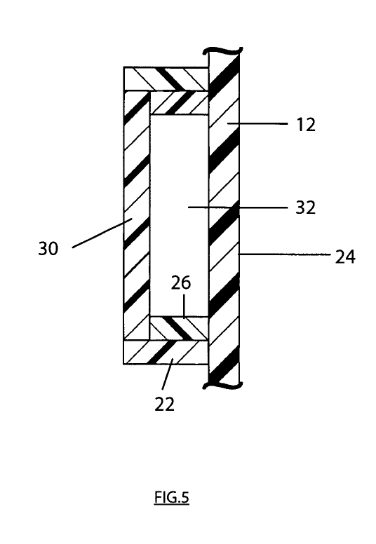

The thermal backing 30 is bonded to the seal 26 so that an air space 32 (best shown in FIG. 5) is created behind the viewing area 24 of the lens 10 between the lens body 12 and the thermal backing 30. The thermal backing 30 has a shape which corresponds to the shape of both the seal 26 and the rib 22 that surrounds the seal. Thus, the thermal backing 30 is spaced from the lens body 12 so as to lie closer to the wearer's eyes than the body 12. A volume of air is trapped along the viewing area 24 within the space 32 between the lens body 12 and thermal backing 30. However, it is within the scope of this invention for thermal insulators (e.g., gases) other than air to be located within the space 32 between the lens body 12 and the thermal backing 30 to prevent the transfer of heat from one to the other. It is also within the scope of this invention for the rib 22 to be molded into and stand outwardly from the thermal backing 30 rather than from the lens body 12 as previously described. In this case, the rib 22 will extend from thermal backing 30 towards the lens body 12.

By virtue of the air space 32 and the air that is trapped therewithin between the lens body 12 and the thermal backing 30, fogging of the viewing area 24 through the protective lens 10 will be avoided so that the wearer's line of sight will not be obstructed. Such fogging could otherwise occur as a consequence of a temperature differential between the lens body 12 which is exposed to the atmosphere and the thermal backing 30 which is pressed against the wearer's face while the lens 10 is being used. Thus, the air (or other gas) within the air space 32 provides a layer of insulation across the viewing area 24. What is more, the seal 26 that is pressed against the rib 22, blocks dirt, water, debris and paint (in situations where the wearer is playing the game of paintball) from entering the air space 32 and reaching the wearer's eyes or covering the thermal backing 30. At the same time, and as an important advantage, the upstanding rib 22 which surrounds the seal 26 safeguards and prevents the seal from becoming saturated with paint and other liquids and degrading over time in order to avoid a possible delamination of the thermal backing 30 from the lens body 12 at the seal. That is to say, the rib 22 shields the seal 26 from the environment and being struck by a high speed paintball and covered with paint should the paintball explode on impact. Likewise, the rib 22 protects the seal 26 from being tampered with and picked off the lens 10 by the wearer's fingernail or other pointed object that might be encountered during play. Thus, the reliability of the seal 26 can be preserved and the life of protective lens 10 maximized.

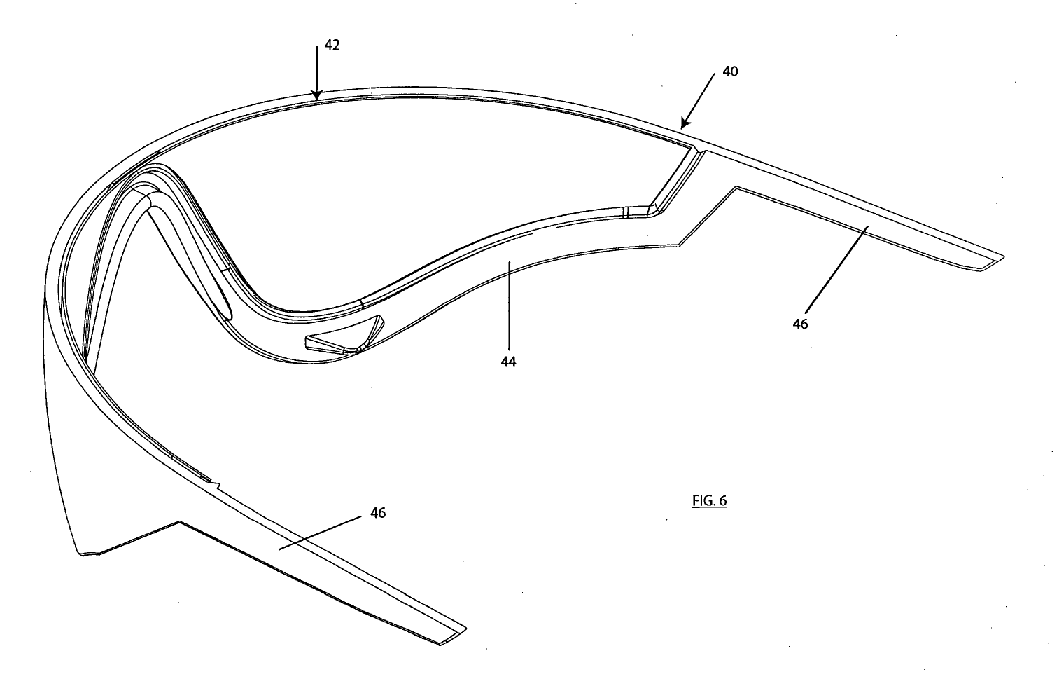

Turning now to FIG. 6 of the drawings, there is shown a pair of glasses 40 having a protective lens 42 according to another preferred embodiment of this invention. As with the protective lens 10 shown in FIGS. 1-5, the protective lens 42 of the glasses 40 of FIG. 6 includes an arcuate lens body 44, the opposite sides 46 of which are held in place by friction behind the ears of the wearer as in the case of a standard pair of glasses. However, the optional attachment holes (designated 18 in FIGS. 2-4) are deleted from the sides 46 of the lens body 44 inasmuch as the mask of FIG. 1 and the headstrap of FIG. 2 are not required. The protective lens 42 is manufactured according to the teachings of this invention with respect to the lens 10 described above.

1. Goggles to protect the eyes of a wearer, comprising:
a transparent lens to cover the eyes of the wearer;
a transparent backing located over said transparent lens and lying closer to the eyes of the wearer than said lens; and
a transparent insulator located between said lens and said backing and adapted to prevent fogging of the lens as a consequence of the transparent lens and the transparent backing having different temperatures.

2. The goggles recited in claim 1, wherein said transparent backing is separated from said transparent lens by a space therebetween, said transparent insulator being located within said space.

3. The goggles recited in claim 2, wherein said space between said transparent backing and said transparent lens is isolated from the atmosphere.

4. The goggles recited in claim 3, wherein said transparent insulator located within said space is air.

5. The goggles recited in claim 3, wherein said space is surrounded by and isolated from said atmosphere by a seal located between said transparent backing and said transparent lens.

6. The goggles recited in claim 5, wherein said seal is manufactured from foam.

7. The goggles recited in claim 5, wherein said seal is attached to said transparent lens and said transparent backing is attached to said seal.

8. The goggles recited in claim 5, further comprising a rib extending between said transparent backing and said transparent lens and surrounding the space therebetween.

9. The goggles recited in claim 8, wherein said rib surrounds and lies outside said seal.

10. The goggles recited in claim 1, wherein said transparent backing is manufactured from a material that is adapted to resist changes in shape and dimension in response to changes in temperature.

11. The goggles recited in claim 1, wherein said transparent lens has an attachment hole formed in each side thereof within which to receive a connector attached to a head strap.

12. A combination, comprising:
a face mask to be worn over the head of a wearer;
a transparent lens carried by said face mask to cover the eyes of the wearer;
a transparent backing separated from said transparent lens and lying closer to the eyes of the wearer than said lens;
a gas trapped in a space between said lens and said backing and adapted to prevent fogging of the lens as a consequence of the transparent lens and the transparent backing having different temperatures; and
a seal located between said transparent backing and said transparent lens and surrounding said space therebetween for isolating said space from the atmosphere.

13. The combination recited in claim 12, further comprising a rib extending between said transparent backing and said transparent lens, said rib surrounding and lying outside said seal so as to block said seal from the atmosphere.

14. The combination recited in claim 13, wherein said rib projects from one of said transparent lens or said transparent backing towards the other, and said seal lies against and inside said rib.

15. Glasses to protect the eyes of a wearer, comprising:
a transparent lens to cover the eyes of the wearer;
a transparent backing separated from said transparent lens and lying closer to the eyes of the wearer than said lens;
a gas trapped in a space between said lens and said backing and adapted to prevent fogging of the lens as a consequence of the transparent lens and the transparent backing having different temperatures; and
a seal located between said transparent backing and said transparent lens and surrounding said space therebetween for isolating said space from the atmosphere.

16. The glasses recited in claim 15, further comprising a rib extending between said transparent backing and said transparent lens, said rib surrounding and lying outside said seal so as to block said seal from the atmosphere.

17. The glasses recited in claim 16, wherein said rib projects from one of said transparent lens or said transparent backing towards the other, and said seal lies against and inside said rib.
